(19) United States
(12) Patent Application Publication
(10) Pub. No.: US 2016/0256457 A1
(43) Pub. Date: Sep. 8, 2016

(54) METHODS FOR TREATING OR PREVENTING ACUTE VASCULAR LEAK

(71) Applicants: Beth Israel Deaconess Medical Center, Inc., Boston, MA (US); The Brigham and Women's Hospital, Inc., Boston, MA (US)

(72) Inventors: Christopher V. CARMAN, Brighton, MA (US); Roberta MARTINELLI, Brighton, MA (US); Thomas MICHEL, Lincoln, MA (US); Hermann KALWA, Brookline, MA (US)

(21) Appl. No.: 14/735,250

(22) Filed: Jun. 10, 2015

Related U.S. Application Data
(60) Provisional application No. 62/010,320, filed on Jun. 10, 2014.

(51) Int. Cl. A61K 31/496; A61K 45/06

(52) U.S. Cl. CPC A61K 31/496; A61K 45/06

(57) ABSTRACT

The invention features methods, devices, pharmaceutical compositions, and kits for treating or preventing acute vascular leak. Such methods may involve contacting an effective amount of bosutinib for treating or preventing acute vascular leak with a subject in need thereof. Conditions associated with acute vascular leak include ischemic brain injury, stroke, septic shock, anthrax lethal toxin, anaphylaxis, trauma, severe episodic vasculitis, acute lung injury, ventilator-induced lung injury, transfusion-related acute lung injury, acute respiratory distress syndrome, severe burn injury, circulatory shock, capillary leak syndrome, and ischemia-reperfusion injury. The invention also features automatic injection devices, resuscitation fluids, or pharmaceutical compositions comprising an effective amount of bosutinib for treating or preventing acute vascular leak. The invention further features kits for use in the methods of the invention.

### STATEMENT AS TO FEDERALLY FUNDED RESEARCH

This invention was made with government support under Grant No. R01HL104006, awarded by the National Institutes of Health (NIH). The government has certain rights in the invention.

### FIELD OF THE INVENTION

The present invention relates to methods, devices, pharmaceutical compositions, and kits for treating or preventing acute vascular leak in subjects.

### BACKGROUND

Blood vessels are lined with tightly linked cells, called endothelial cells, which normally form a largely impermeable barrier. Vascular leak occurs when small blood vessels, generally a capillary or venule, become leaky and release fluid and plasma proteins. The most serious effects of vascular leak include a drop in blood pressure, severe organ damage, and a lack of oxygenation of the blood when the leak is in the lung. Vascular leak contributes to wide ranging inflammatory pathologies in ways that dramatically influence outcomes. Therapeutic strategies that directly act at the level of vascular leak are non-existent and could have profound effects, particularly for acute onset pathologies such as sepsis, traumatic shock, ischemia reperfusion and exposure to anthrax Lethal Toxin.

In the case of sepsis, initial systemic infection leads to a rapid inflammatory response termed a “cytokine storm” that causes to a rapid breakdown of vascular integrity. The ensuing vascular leak, which is evident in experimentally induced models of sepsis within ˜6-12 hours, plays a central role in creating life-threatening cardiovascular dysfunction, tissue damage and organ failure. Sepsis accounts for 3% of all admissions to U.S. hospitals, generates annual direct costs in excess of $16 billion, and is associated with an acute mortality of ˜30%. Death in sepsis is due to septic shock and multi-organ dysfunction. When patients present in the Emergency Department (ED), septicemia, cytokine storm and vascular leak are typically well underway. During the critical 12-48 hours following presentation in the ED, patients are highly vulnerable to the downstream consequence of vascular leak and edema. However, frontline strategies are limited to antibiotics and fluid resuscitation.

Capillary permeability, which is a tightly regulated feature of microcirculation in all organ beds, is fundamentally altered during sepsis, resulting in net extravasation of fluid and protein out of the vascular space and into tissues. A dramatic manifestation of this phenomenon is acute lung injury (ALI) and its sequela, acute respiratory distress syndrome (ARDS), which occurs in up to 40% of patients with sepsis. ARDS is marked by leakage of fluid out of pulmonary capillaries and into alveolar septa and air spaces. Excess extravascular fluid in the lung impairs gas exchange across the alveolar membrane and decreases lung compliance. In ARDS, small blood vessels in the lungs become leaky and release fluid/protein, resulting in impaired lung function. Often, the damage becomes extensive enough to necessitate the use of mechanical ventilation. If the condition lasts too long, the lung tissue gets damaged irreversibly and ARDS associated with sepsis has been correlated with a mortality of 40%.

There is currently no therapeutic strategy to directly address vascular leak pathologies such as sepsis, and it is widely believed that a strategy for acute protection/rescue of vascular integrity and barrier function could have profound effect of clinical outcomes. Indeed, a recent NIH Working Group of inter-disciplinary scientific investigators held in June 2010, concluded that: “Sepsis poses a serious public health problem in the United States and globally with an overall mortality rate of 30%. Our current understanding of sepsis expands the cytokine storm paradigm′ to the unifying concept of severe endothelial dysfunction syndrome in response to intravascular or extravascular microbial agents that cause multi-organ failure. It is therefore clear that breakdown of the blood/endothelial tissue barrier is one of the major contributors to sepsis morbidity and mortality.” Despite extensive efforts, sepsis outcomes have not substantially improved over the past thirty years.

Bosutinib (SKI-606, Bosulif©) was identified in a screen against Src kinase and later shown to potently inhibit c-Abl. Bosutinib is now indicated for the treatment of adult patients with chronic, accelerated, or blast phase Philadelphia chromosome positive (Ph+) chronic myelogenous leukemia (CML) with resistance or intolerance to prior therapy. Bosutinib is a member of the BCR-Abl tyrosine kinase inhibitor (TKI) family. TKIs have become the first-line therapy for most CML patients. Gleevec© (i.e., Imatinib, STI571) was discovered in 1992 and is regarded as a first generation drug since it was the first BCR-Abl tyrosine kinase inhibitor to be used in the treatment of chronic CML 1. Following emergence of patient populations exhibiting resistance to Imatinib, a range of second generation TKIs have been developed for CML, including Bosutinib, Dasatinib (BMS-345825, Sprycel©), Nilotinib (AMN107, Tasigna©), Ponatinib (AP24534, Iclusig©), and Bafetinib (INNO-406, NS187), which together with Imatinib are all generally thought to act through dual specific inhibition of Abl and Src-family kinases.

In summary, new strategies are urgently required for supporting vascular integrity/barrier function. Thus, there remains a clear need in the art for methods to treat or prevent vascular leak and conditions associated with vascular leak.

### SUMMARY OF THE INVENTION

The invention features methods, pharmaceutical compositions, and kits for treating or preventing acute vascular leak in subjects in need thereof. Acute vascular leak treatable or preventable with embodiments of the present invention include, without limitation, ischemic brain injury, stroke, septic shock, anthrax lethal toxin, anaphylaxis, trauma, severe episodic vasculitis, acute lung injury, ventilator-induced lung injury, transfusion-related acute lung injury, acute respiratory distress syndrome, severe burn injury, circulatory shock, capillary leak syndrome, and ischemia-reperfusion injury.

In a first aspect, the invention features a method of treating or preventing acute vascular leak in a subject in need thereof, in which the method involves contacting an effective amount of bosutinib with the subject, and in which the acute vascular leak is associated with a condition selected from the group consisting of: ischemic brain injury, septic shock, anthrax lethal toxin, anaphylaxis, trauma, severe episodic vasculitis, acute lung injury, acute respiratory distress syndrome, severe burn injury, circulatory shock, capillary leak syndrome, and ischemia-reperfusion injury.

In a preferred embodiment, the condition is septic shock.

In particular embodiments, the ischemic brain injury is stroke.

In certain embodiments, the acute lung injury is ventilator-induced lung injury or transfusion-related acute lung injury.

In other embodiments, the trauma is selected from the group consisting of: severe tissue trauma, head trauma, lung trauma, cardiac trauma, and vascular trauma.

In certain embodiments, the subject is at risk for acute vascular leak associated with a condition selected from the group consisting of: ischemic brain injury, septic shock, anthrax lethal toxin, anaphylaxis, trauma, severe episodic vasculitis, acute lung injury, acute respiratory distress syndrome, severe burn injury, circulatory shock, capillary leak syndrome, and ischemia-reperfusion injury.

In some embodiments, the bosutinib is administered by autoinjection, as a component of a resuscitation fluid, sublingually, or by inhalation.

In certain embodiments, the method further includes administering a second therapeutic agent.

In a preferred embodiment, the second therapeutic agent includes a Src inhibitor.

In some embodiments, the Src inhibitor is selected from the group consisting of: Nilotinib, Ponatinib, Bafetinib, imatinib, dasatinib, PP1, PP2, sunitinib, and SKS-927. In certain embodiments, the Src inhibitor is Nilotinib, Ponatinib, Bafetinib, sunitinib, or SKS-927.

In other embodiments, the second therapeutic agent includes a Rho kinase inhibitor.

In some embodiments, the Rho kinase inhibitor is Y-27632 or fasudil.

In other embodiments, the second therapeutic agent includes a soluble epoxide hydrolase inhibitor. In certain embodiments, the soluble epoxide hydrolase inhibitor is 12-(3-adamantan-1-yl-ureido)-dodecanoic acid butyl ester (AU DA-BE), 1-adamantan-3-(5-(2-(2-ethyl-ethoxy)ethoxy)pentyl)urea (compound 950), and/or any combination thereof.

In some embodiments, the soluble epoxide hydrolase inhibitor includes AUDA-BE and compound 950.

In other embodiments, the second therapeutic agent includes a TREM1 inhibitor.

In some embodiments, the TREM1 inhibitor is recombinant TREM-like transcript 1 (TLT-1), LR17 peptide, Inotrem MOTREM peptide, or SignaBlock SCHOOL peptide.

In other embodiments, the second therapeutic agent includes a TNF-α inhibitor.

In some embodiments, the TNF-α inhibitor is selected from the group consisting of: infliximab, adalimumab, etanercept, and ADZ9773.

In some embodiments, the second therapeutic agent is selected from the group consisting of: an antibiotic, drotrecogin alpha, a corticosteroid, vasopressin, a mechanical ventilation device, surgical drainage of infected fluid collections, fluid replacement, support for organ dysfunction (e.g., hemodialysis in kidney failure, mechanical ventilation in pulmonary dysfunction, transfusion of blood plasma, platelets, and coagulation factors to stabilize blood coagulation, or drug and fluid therapy for circulatory failure), activated protein C therapy (e.g., drotrecogin alfa), corticosteroid treatment, vasopressin, inhibitors of MLC kinase, inhibitors of VEGF (e.g., avastin), inhibitors of PIGF, inhibitors of NFκB (e.g., panepoxydone), inhibitors of TNF-α, inhibitors of IL-1, inhibitors of IL-6, Ang-2 antagonists, and inhibitors of TGF-β.

In a second aspect, the invention features a method of treating vascular leak in a subject in need thereof. The method involves:
(i) identifying a subject that will likely benefit from bosutinib treatment (e.g., bosutinib treatment according to any of the methods of the invention) by, for example, monitoring one or more measures reflective of vascular leak (e.g., exudative vascular leak and/or acute vascular leak), such as by measuring blood pressure, edema (e.g., conducting physical examination of measure ankle thickness and/or tissue swelling) and/or severity of organ dysfunction (e.g., by conducting one or more Sequential Organ Failure Assessments (SOFA) via, for example, standardized laboratory tests and clinical data, e.g., measuring bilirubin, creatinine, platelet count, partial pressure of oxygen, fraction of inhaled O₂, the Glasgow Coma Scale, and/or hypotension), or any combination thereof; and
(ii) if the one or more measures indicate increased vascular leak (e.g., by a decrease in blood pressure, an increase in edema [e.g., as indicated by increased ankle thickness and/or increased tissue swelling], an increase in severity of organ dysfunction, or any combination thereof), administering an effective amount of bosutinib to the subject, thus treating the vascular leak in the subject.

In certain embodiments, the method further includes monitoring the one or more measures reflective of vascular leak (e.g., measuring blood pressure, edema [e.g., by conducting physical examination of ankle thickness and/or tissue swelling], severity of organ dysfunction, or any combination thereof) after the administration, in which a reduction of the indication of increased vascular leak (e.g., an increase in blood pressure, a decrease in edema [e.g., as indicated by decreased ankle thickness and/or decreased tissue swelling], a decrease in severity of organ dysfunction, or any combination thereof) indicates treatment of the vascular leak.

In various embodiments of the second aspect, the one or more measures reflective of vascular leak (e.g., the one or more measures used to identify a subject that will likely benefit from bosutinib treatment, and/or the one or more measures monitored) include measuring red blood cell (RBC) velocity, RBC supply rate, percent oxygen saturation, and/or capillary diameter. In certain embodiments, the indication of increased vascular leak (e.g., the indication of increased vascular leak used to identify a subject that will likely benefit from bosutinib treatment, and/or the indication of increased vascular leak monitored) includes a decrease in RBC velocity, a decrease in RBC supply rate, decrease in oxygen saturation, or any combination thereof.

In some embodiments, the bosutinib has not been previously administered to the subject to treat the vascular leak.

In other embodiments, the bosutinib has been previously administered to the subject to treat the vascular leak.

In some embodiments, the vascular leak is an acute vascular leak.

In certain embodiments, the acute vascular leak is associated with a condition selected from the group consisting of: ischemic brain injury, stroke, septic shock, anthrax lethal toxin, anaphylaxis, trauma, severe episodic vasculitis, acute lung injury, ventilator-induced lung injury, transfusion-related acute lung injury, acute respiratory distress syndrome, severe burn injury, circulatory shock, capillary leak syndrome, and ischemia-reperfusion injury.

In some embodiments, the bosutinib is administered by autoinjection, as a component of a resuscitation fluid, sublingually, or by inhalation.

In certain embodiments of any of the above aspects, the subject has previously been treated with a Src inhibitor and the vascular leak has not improved after the treatment with the Src inhibitor. Vascular leak can be evaluated using standard methods in the art, including but not limited to monitoring blood pressure, edema (e.g., conducting physical examination of measure ankle thickness and/or tissue swelling) and/or severity of organ dysfunction (e.g., by conducting Sequential Organ Failure Assessments (SOFA) via, for example, standardized laboratory tests and clinical data, e.g., measuring bilirubin, creatinine, platelet count, partial pressure of oxygen, fraction of inhaled O₂, the Glasgow Coma Scale, and/or hypotension). Improvement in vascular leak can be shown, for example, by an increase in blood pressure, a decrease in edema (e.g., as indicated by decreased ankle thickness and/or decreased tissue swelling), a decrease in severity of organ dysfunction, or any combination thereof,

In some embodiments, Src inhibitor is selected from the group consisting of: Nilotinib, Ponatinib, Bafetinib, imatinib, dasatinib, PP1, PP2, sunitinib, and SKS-927. In certain embodiments, the Src inhibitor is Nilotinib, Ponatinib, Bafetinib, sunitinib, or SKS-927.

In an embodiment of any of the above aspects, the subject is not suffering from leukemia.

In a third aspect, the invention features an automatic injection device including a pre-loaded charge of medicament for automatically self-administering the medicament upon actuation thereof, in which the medicament includes an effective amount of bosutinib for treating or preventing vascular leak.

In another aspect, the invention features a resuscitation fluid including an effective amount of bosutinib for treating or preventing acute vascular leak.

In certain aspects, the invention features pharmaceutical compositions including a tablet, liquid, or powder to be administered sublingually, in which the tablet, liquid, or powder includes an effective amount of bosutinib for treating or preventing acute vascular leak.

In another aspect, the invention features a pharmaceutical composition including an aerosol, in which the aerosol includes an effective amount of bosutinib for treating or preventing acute vascular leak.

In some embodiments, the acute vascular leak is associated with a condition selected from the group consisting of: ischemic brain injury, stroke, septic shock, anthrax lethal toxin, anaphylaxis, trauma, severe episodic vasculitis, acute lung injury, ventilator-induced lung injury, transfusion-related acute lung injury, acute respiratory distress syndrome, severe burn injury, circulatory shock, capillary leak syndrome, and ischemia-reperfusion injury.

In another aspect, the invention features a kit for treating or preventing acute vascular leak in a subject in need thereof, in which the kit includes an effective amount of bosutinib and instructions for administering the bosutinib to the subject.

In some embodiments, the kit includes an automatic injection device, in which the automatic injection device includes the bosutinib.

In certain embodiments, the kit includes a resuscitation fluid, in which the resuscitation fluid includes the bosutinib.

In particular embodiments, the kit includes a tablet, liquid, or powder to be administered sublingually, in which the tablet, liquid, or powder includes the bosutinib.

In further embodiments, the kit includes an aerosol, in which the aerosol includes the bosutinib.

In some embodiments, the acute vascular leak is associated with a condition selected from the group consisting of: ischemic brain injury, stroke, septic shock, anthrax lethal toxin, anaphylaxis, trauma, severe episodic vasculitis, acute lung injury, ventilator-induced lung injury, transfusion-related acute lung injury, acute respiratory distress syndrome, severe burn injury, circulatory shock, capillary leak syndrome, and ischemia-reperfusion injury.

### DEFINITIONS

As used herein, the terms “bosutinib” or “SKI-606” mean a Bcr-Abl tyrosine kinase inhibitor compound with the following chemical structure:

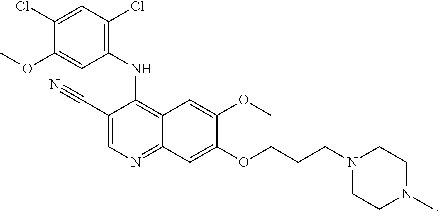

By “compound,” as used herein, is meant to include all stereoisomers, geometric isomers, tautomers, and isotopes of the structures depicted. Compounds of the present disclosure also include all of the isotopes of the atoms occurring in the compounds. “Isotopes” refers to atoms having the same atomic number but different mass numbers resulting from a different number of neutrons in the nuclei. For example, isotopes of hydrogen include tritium and deuterium.

The compounds and salts of the present disclosure can be prepared in combination with solvent or water molecules to form solvates and hydrates by routine methods.

By “administered in combination” or “combined administration” is meant that two or more agents are administered to a subject at the same time or within an interval such that there may be an overlap of an effect of each agent on the patient. In some embodiments, they are administered within about 60, 30, 15, 10, 5, or 1 minute of one another. In some embodiments, the administrations of the agents are spaced sufficiently closely together such that a combinatorial (e.g., a synergistic) effect is achieved.

The term “pharmaceutical composition,” as used herein, represents a composition containing an agent or compound described herein formulated with a pharmaceutically acceptable excipient. In some embodiments, the pharmaceutical composition is manufactured or sold with the approval of a governmental regulatory agency as part of a therapeutic regimen for the treatment of disease in a mammal. Pharmaceutical compositions can be formulated, for example, for intramuscular administration; administration as a component of a resuscitation fluid, sublingual administration (e.g., as a tablet, liquid, or powder to be placed under the tongue and absorbed sublingually); for administration by inhalation; for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

A “pharmaceutically acceptable excipient,” as used herein, refers any ingredient other than the compounds described herein (for example, a vehicle capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, or waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

In addition, compounds of the invention may be coupled through conjugation to substances designed to alter the pharmacokinetics, for targeting, or for other reasons. Thus, the invention further includes conjugates of these compounds. For example, polyethylene glycol is often coupled to substances to enhance half-life; the compounds may be coupled to liposomes covalently or noncovalently or to other particulate carriers. They may also be coupled to targeting agents such as antibodies or peptidomimetics, often through linker moieties. Thus, the invention is also directed to compounds when modified so as to be included in a conjugate of this type.

By “condition,” “disorder,” or “disease” is meant any state that would benefit from treatment with, for example, a substance, compound, pharmaceutical composition, method, or kit of the invention. This includes chronic and acute disorders or diseases including those pathological conditions which predispose the subject to the disorder in question. Non-limiting examples of conditions to be treated herein include ischemic brain injury, stroke, septic shock, anthrax lethal toxin, anaphylaxis, trauma, severe episodic vasculitis, acute lung injury, ventilator-induced lung injury, transfusion-related acute lung injury, acute respiratory distress syndrome, severe burn injury, circulatory shock, capillary leak syndrome, and ischemia-reperfusion injury.

As used herein, the terms “subject” or “patient” refer to any organism to which a compound or composition in accordance with the invention may be administered, e.g., for experimental, diagnostic, prophylactic, and/or therapeutic purposes. Typical subjects include animals (e.g., mammals such as mice, rats, rabbits, non-human primates, and humans) and/or plants.

By “preventing” or “prevention” is meant prophylactic treatment of a subject who is not yet ill, but who is susceptible to, or otherwise at risk of, developing a particular disease. Preferably, a subject is determined to be at risk of developing any type of vascular leak, for example resulting from sepsis or interleukin-2 therapy, using the diagnostic methods known in the art or described herein. For example, in the case of a patient already diagnosed with sepsis, “preventing” can refer to the prevention of severe sepsis, lung failure, or death. In another example, in the case of a patient undergoing IL-2 therapy, prevention can refer to prevention of the onset of vascular leak. In another example, in subjects undergoing blood transfusion or mechanical ventilation, prevention can refer to prevention of the onset of vascular leak resulting from transfusion-related acute lung injury or ventilator-induced lung injury, respectively. In certain embodiments, prevention of, e.g., the onset of vascular leak is achieved by administration of an effective amount of bosutinib to the subject prior to and/or concurrent with the blood transfusion or mechanical ventilation.

The term “effective amount” of an agent, as used herein, is that amount sufficient for the agent to effect beneficial or desired results, such as clinical results, and, as such, an “effective amount” depends upon the context in which it is being applied.

By “treating” or “treatment” is meant administering a compound or a pharmaceutical composition for prophylactic and/or therapeutic purposes or administering treatment to a subject already suffering from a disease to improve the subject's condition or to a subject who is at risk of developing a disease. By “treating a vascular leak disorder” is meant that the disease and the symptoms associated with the disease are alleviated, reduced, cured, or placed in a state of remission. More specifically, when bosutinib, or a pharmaceutical composition comprising bosutinib, is used to treat a subject with a vascular leak disorder, it is generally provided in a therapeutically effective amount to achieve any one or more of the following: reduce mortality, reduce vascular leakage, restore the integrity of vessel walls, prevent requirement for mechanical ventilation, reduce organ damage, increase in arterial blood pressure, increase in cardiac output, decreased systemic vascular resistance, decrease in the number of vasopressor medications necessary to maintain tissue perfusion, reduction in edema-bedside clinical assessment, increased urine output, decreased weight gain upon administration of intravenous fluids, increase in oxygenation of blood-increased PaO2/FiO2, increased oxygen saturation (SpO2), decreased positive end-expiratory pressure (PEEP) needed to ventilate lungs adequately, fall in respiratory rate, decrease in time to discontinuing mechanical ventilation, decrease in number of ICU days required, and decrease in time to resolution of shock.

As used herein, the term “resuscitation fluid” refers to a solution for use in intervention in acute medicine, which may be introduced to a subject, e.g., intravenously. Examples of resuscitation fluids include, but are not limited to, colloid solutions, crystalloid solutions, albumin solution, hydroxyethyl starch solution, succinylated modified fluid gelatin, urea-linked gelatin, balanced salt solutions, 0.9% saline, and compounded sodium lactate.

By “vascular leak” is meant the movement of fluid, plasma proteins, blood cells, and/or other blood constituents from the blood vessels into the surrounding tissues. Symptoms of vascular leak include reduced blood pressure, reduced cardiac output, increased systemic vascular resistance, edema, decreased urine output, decreased blood oxygenation, increase in respiratory rate, and shock.

By “acute vascular leak” is meant any vascular leak with a rapid onset and/or a short course, for example, acute vascular leak associated with any one or more of the following conditions: ischemic brain injury, stroke, septic shock, anthrax lethal toxin, anaphylaxis, trauma, severe episodic vasculitis, acute lung injury, ventilator-induced lung injury, transfusion-related acute lung injury, acute respiratory distress syndrome, severe burn injury, circulatory shock, capillary leak syndrome, and ischemia-reperfusion injury.

By “vascular leak condition,” “condition associated with vascular leak,” “vascular leak pathology,” “vascular leak disorder,” or “vascular leak disease” is meant any disease, disorder, or condition that is characterized by a vascular leak or has associated with it the presence of a vascular leak. Vascular leak conditions include, for example, any of the following disorders that have associated vascular leak: sepsis (e.g., mild or severe), septic shock, anthrax, pneumonia, acute lung injury (ALI; e.g., ventilator-induced lung injury or transfusion-related acute lung injury), acute respiratory distress syndrome (ARDS; e.g., transplant ARDS and ARDS due to burns, pancreatitis and trauma), capillary leak syndrome, ischemic brain injury, anaphylaxis, severe episodic vasculitis, ischemia-reperfusion injury, vascular leak associated with drug therapy (e.g., IL-2, IL-11, P277, ibuprofen, aspirin, salicylic acid derivatives, anagrelide, cilostazol, clopidogrel, dipyridamole, ticlopidine, anisindione, enoxaparin, heparin, pentosan polysulfate, aceclofenac, warfarin, acenocoumarol, non-steroidal anti-inflammatory drugs, selective serotonin reuptake inhibitors, and others), anthrax lethal toxin, idiopathic capillary leak syndromes, pre-eclampsia, eclampsia, hypotensive states due to sepsis, heart failure, severe burn injury, circulatory shock, trauma, infection, pulmonary aspiration of stomach contents, pulmonary aspiration of water, near drowning, burns, inhalation of noxious fumes, fat embolism, blood transfusion (TRALI, transfusion-related acute lung injury), amniotic fluid embolism, air embolism, edema, organ failure, poisoning, radiation, and inflammatory states (e.g., inflammation associated with acute and chronic vascular rejection, pancreatitis, trauma, or vasculitis). Less common etiologies of vascular leak include genetic disorders that intermittently produce vascular leak (e.g. C1 esterase inhibitor deficiency or familial fever syndromes such as TRAPS-TNF receptor associated periodic fever syndrome), massive blood transfusion, anaphylaxis or similar hypersensitivity reactions, post-lung or post-heart-lung transplant, and ovarian hyperstimulation syndrome (e.g., as described in Garcia-Velasco et al. Curr Opin Obstet Gynecol. 15:251-256, 2003). Vascular leak can also be caused by VEGF or bradykinin overexpression.

By “Src inhibitor” is meant an agent such as, e.g., a compound, antibody, polypeptide, or nucleic acid, that inhibits the activity of a Src protein. Src proteins are a family of mammalian cytoplasmic tyrosine kinases that play an extensive role in signal transduction and include, for example, Src, Yes, Fyn, c-Src, v-Src, Fgr, Lck, Hck, Blk, Lyn, and Frk. Examples of Src inhibitors include but are not limited to: bosutinib, imatinib, sunitinib, PP1(1-(1,1-dimethylethyl)-1-(4-methylphenyl)-1H-pyrazolo[3,4-d]pyrimidin-4-amine), PP2 (3-(4-chlorophenyl)-1-(1,1-dimethylethyl)-1H-pyr-azolo[3,4-d]pyrimidin-4-amine), damnacanthal (3-hydroxy-1-methoxy-2-anthra-quinonecarboxaldehyde), SKS-927, and SU-5565.

The term “Rho kinase inhibitor” refers to an agent such as, e.g., a compound, antibody, polypeptide, or nucleic acid, that inhibits the activity of a Rho kinase protein such as ROCK1 or ROCK2. Examples of Rho kinase inhibitors include but are not limited to: Y-27632, fasudil, hydroxyfasudil Y-39983, Wf-536, SLx-2119, azabenzimidazole-aminofurazan, DE-104, olefin, isoquinoline, indazole, H-1152P, XD-4000, HMN-1152, and rhostatin.

By “soluble epoxide hydrolase inhibitor,” “epoxide hydrolase inhibitor,” or “sEH inhibitor” is meant an agent such as, e.g., a compound, antibody, polypeptide, or nucleic acid, that inhibits the activity of the sEH protein. The sEH protein is crucial to the formation of the active lipid mediator Leukocytoxin-Diol from a Leukotoxin precursor by neutrophils during systemic inflammation. Leukocytoxin-Diol is known to induce vascular leak and death and is critical for ARDS. sEH block formation of Leukocytoxin-Diol and may be effective in preventing ARDS in settings of sepsis and severe burn, as shown in animal studies. Examples of sEH inhibitors include but are not limited to: 12-(3-adamantan-1-yl-ureido)-dodecanoic acid butyl ester (AU DA-BE) and 1-adamantan-3-(5-(2-(2-ethyl-ethoxy)ethoxy)pentyl)urea (compound 950).

The term “TREM1 inhibitor” refers to an agent such as, e.g., a compound, antibody, polypeptide, or nucleic acid, that inhibits the activity of the TREM1 protein. TREM1 is a major mediator of neutrophil activation/neutrophil-mediated endothelial cell injury. TREM1 inhibitors have shown efficacy in treating such neutrophil-mediated endothelial cell injury in murine sepsis models. Examples of TREM1 inhibitors include but are not limited to: recombinant TREM-like transcript 1 (TLT-1), LR17 peptide, Inotrem MOTREM peptide and SignaBlock SCHOOL peptide.

By “TNF-α inhibitor” or “TNF inhibitor” is meant an agent such as, e.g., a compound, antibody, polypeptide, or nucleic acid, that inhibits the activity of the TNF-α protein. TNF inhibitors are known candidate drugs for treating sepsis. Examples of TNF-α inhibitors include, but are not limited to, infliximab, adalimumab, etanercept and ADZ9773.

As used herein, the term “trauma” refers to a physical wound to the body or to a part of the body. Trauma can, e.g., be associated with inflammatory responses, including but not limited to systemic inflammation and cytokine storm. Non-limiting examples of trauma include blunt trauma, burn injury, head trauma (e.g., facial trauma), lung trauma, cardiac trauma, abdominal trauma, chest trauma, polytrauma, limb trauma (e.g., trauma to the arms, legs, hands, feet, and/or digits), muscle trauma, bone trauma, airway trauma, blood vessel trauma, nervous system trauma (e.g., spinal cord injury, brain injury, or peripheral nerve injury), gastrointestinal trauma, or kidney trauma.

By the term “barrier leak correction” is meant treatment of vascular leak associated with impaired barrier function (e.g., reduced barrier integrity and/or increased vascular permeability). Exemplary methods for barrier leak correction include methods of treating or preventing vascular leak (e.g., acute vascular leak) such as those described herein, treatment with classic barrier protectors (e.g., adrenomedulin, hepatocyte growth factor, angiopoietin-1, sphingosine-1-phosphates, beta-agonists [e.g., isoproterenol], activated protein C (APC), O-methyl-cAMP, Fausudil, and Y27632), or any combination thereof.

As used herein, “classic barrier protectors” refer to compounds and/or agents known to be useful for treating and/or preventing conditions associated with vascular leak. Non-limiting examples of classic barrier protectors include: adrenomedulin, hepatocyte growth factor, angiopoietin-1, sphingosine-1-phosphates, beta-agonists (e.g., isoproterenol), activated protein C (APC), O-methyl-cAMP, fasudil, and Y27632.

By “support for organ dysfunction” is meant any medical intervention intended to treat, prevent, and/or mitigate organ dysfunction. Non-limiting examples of support for organ dysfunction include hemodialysis (e.g., for kidney failure); mechanical ventilation (e.g., for pulmonary dysfunction); transfusion of blood plasma, platelets, and coagulation (e.g., to stabilize blood coagulation), and drug and fluid therapy (e.g., for circulatory failure).

### BRIEF DESCRIPTION OF THE DRAWINGS

FIG. 1 is a graph showing that bosutinib, but not the related compounds imatinib and dasatinib, promotes increase in vascular barrier function in vitro. Primary human dermal microvascular endothelial cells were grown to confluence. Steady state barrier function was monitored by Electrical Cell-substrate Impedance Sensing (ECIS) for 24 hours and normalized to a value of 1. As indicated (vertical arrow), single 300 nM doses of bosutinib (Bosu; circles), imatinib (Ima; squares) dasatinib (Dasa; diamonds), or vehicle control (Con; triangles) were added and resistance was monitored for an additional 3 hours. Data are mean±SEM of three independent experiments. Observed increases in resistance following addition of bosutinib are a direct reflection of increased vascular integrity and barrier function. Conversely the observed decreases in resistance following dasatinib addition reflect decreased vascular integrity and barrier function. The unchanged resistance following addition of imatinib indicates no change in barrier function.

FIG. 2 is a graph showing that bosutinib promotes sustained, dose-dependent increase in vascular barrier function in vitro. Primary bovine aortic endothelial cells were grown to confluence. Steady state barrier function was monitored by ECIS for 24 hours and normalized to a value of 1. Single doses of bosutinib were added to yield the indicated final concentrations ranging from 10 nM to 3 μM, and resistance was continuously monitored for an additional 72 hours. Observed increases in resistance are a direct reflection of increased vascular integrity and barrier function. Data are mean±SEM of three independent experiments. The magnitude and duration of these increases was strongly dependent on dose of bosutinib provided.

FIGS. 3A and 3B are graphs showing that bosutinib, but not imatinib, protects vascular barrier function in vitro. FIG. 3A shows the results of thrombin-induced vascular permeability assays while FIG. 3B shows those of histamine-induced vascular permeability assays. For all assays, primary human dermal microvascular endothelial cells were grown to confluence. Steady state barrier function was monitored by ECIS and normalized to a value of 1. Single 300 nM doses of bosutinib (squares), imatinib (triangles) or vehicle control (circles) were added for 30 min followed by addition of permeability inducing agents thrombin (2.5 U/ml; FIG. 3A) or histamine (300 μM; FIG. 3B). Whereas bosutinib provided profound ˜50% and 100% protection to thrombin and histamine challenges, respectively, imatinib yielded a negligible trend of ˜5-10% protection. Data are mean±SEM of three independent experiments.

FIGS. 4A and 4B are graphs showing that bosutinib, but not imatinib or dasatinib, promotes restoration/recovery of vascular barrier function in vitro. FIG. 4A shows the results of thrombin-induced vascular permeability assays while FIG. 4B shows those of histamine-induced vascular permeability assays. For all assays, primary human dermal microvascular endothelial cells were grown to confluence. Steady state barrier function was monitored by ECIS and normalized to a value of 1. Single doses of thrombin (Thr; 2.5 U/ml) or histamine (His; 300 μM) were added to induce a transient decrease in barrier function. After a period of 20-30 minutes, when a ˜25% decrease in resistance was observed, 300 nM doses of bosutinib (Bosu; squares), imatinib (Ima; triangles), dasatinib (Das; diamonds) or vehicle control (Con; circles) were added. In vehicle treated samples, the acute decrease in barrier function was followed by a natural, somewhat slower phase, of barrier recovery back to baseline. Bosutinib was observed to accelerate barrier recovery relative to controls, while imatinib had no effect and dasatinib caused exacerbated and sustained barrier decrease. Data are mean±SEM of three independent experiments.

FIG. 5A is a series of fluorescence microscopy images showing that bosutinib stabilizes endothelial junctions and prevents gap formation during vascular permeability challenge in vitro. Primary human dermal microvascular endothelial monolayers were challenged with either Thrombin (2.5 U/ml) or Histamine (300 μM) for 30 min, with or without 30 min pretreatment with 300 nM doses of bosutinib. Samples were fixed and stained for actin (i) and the junctional marker VE-cadherin (ii) before imaging by confocal microscopy. To assess quality of endothelial junctions (see B), image thresholding was applied to the VE-cadherin images to delineate the junctions (line traces in [iii]). Representative images show that Thrombin and Histamine challenges generate discontinuous junctions with extensive gaps, whereas bosutinib maintains continuous junctions and cortical actin during challenge. Scale bar represents 10 μm.

FIG. 5B is a set of graphs demonstrating quantitatively that bosutinib stabilizes endothelial junctions during vascular permeability challenge, as determined using two readouts for junctional/barrier quality. Primary human dermal microvascular endothelial monolayers were challenged with either Thrombin (Thr) or Histamine (His) as described above for FIG. 5A. Quantitative analysis of (i) total junctional area per field and (ii) VE-cadherin intensity within junctions were performed to assess maintenance of junctional integrity. Bosutinib (Bosu) significantly increased junctional area compared to vehicle controls in each case. Data are mean±SEM of three independent experiments.

FIG. 6 is a graph showing that bosutinib promotes greater overall endothelial barrier enhancement in vitro, compared to benchmark agents. Primary human dermal microvascular endothelial cells were grown to confluence. Steady state barrier function was monitored by ECIS for 24 hours and normalized to a value of 1. As indicated (vertical arrow), single doses of bosutinib (BOSU; triangles; 300 nM), hepatocyte growth factor (HGF; squares; 100 ng/ml, 1200 nM) or activated protein C (APC; circles; 500 nM) were added and barrier function was monitored for an additional ˜18 hours. Bosutinib was observed to promote a significant and sustained increase in barrier function. HGF initially promoted a similar increase, which was rapidly reversed after ˜20 minutes leading ultimately to a sustained decrease in barrier function. APC was observed to promote an increase in barrier function that was relatively minor in both magnitude and duration. Data are mean±SEM of three independent experiments.

FIG. 7 is a graph showing that bosutinib enhances endothelial barrier function, whereas other Src inhibitors, including dasatinib and PP2, inhibit barrier function. Primary human dermal microvascular endothelial cells were grown to confluence. Steady-state barrier function was monitored by ECIS for 24 hours and normalized to a value of 1. As indicated (vertical arrow), single 300 nM doses of Bosutinib (Bosu; triangles; 300 nM), Dasatinib (Das; squares; 300 nM) or PP2 (circles; 10 μM) were added, and barrier function was monitored for an additional ˜22 hours. Whereas bosutinib caused a sustained increase in barrier function, dasatinib and PP2 caused sustained decreases in barrier function. Data are mean±SEM of three independent experiments.

FIGS. 8A-8D are a series of graphs of microvascular functional and structural parameters measured in an established model of polymicrobial sepsis (feces-induced peritonitis; FIP) with either no treatment (drug vehicle (citrate buffer) only; control) or with bosutinib treatment. Disease onset was initiated by tail vein injection of a solution of saline and rat feces. At 30 minutes (‘baseline’ time point) following FIP onset vehicle or a 10 mg/kg loading dose of bosutinib was administered by tail vein injection. Subsequently, maintenance doses of vehicle of 5 mg/kg bosutinib were administered every two hours. Microvascular parameters were measured through intravital microscopy of the hindlimb at baseline, 3 hr and 5 hr post FIP onset. FIGS. 8A and 8B are graphs showing that red blood cell (RBC) velocity and supply rate, respectively (both measures of blood flow efficiency that are altered by vascular leak), rapidly decline following sepsis onset in control rats, but not in bosutinib-treated rats. FIG. 8C is a graph showing that percent oxygen saturation, a critical parameter altered by vascular leak, is similarly maintained in bosutinib-treated, but not control, FIP rats. FIG. 8D is a graph showing that capillary diameter is unchanged in both control and bosutinib rats during FIP onset, indicating that the microvascular function preservation conferred by bosutinib is not attributable to alterations in vasodilation, but rather is attributable to inhibition of vascular leak. Data are representative of two separate pilot experiments.

### DETAILED DESCRIPTION OF THE INVENTION

The present invention provides methods, devices, compositions, and kits for treating and/or preventing vascular leak (e.g., acute vascular leak). Exemplary vascular leak conditions that can be treated or prevented by the methods, devices, compositions and/or kits disclosed herein include, but are not limited to, ischemic brain injury, stroke, septic shock, anthrax lethal toxin, anaphylaxis, trauma, severe episodic vasculitis, acute lung injury, ventilator-induced lung injury, transfusion-related acute lung injury, acute respiratory distress syndrome, severe burn injury, circulatory shock, capillary leak syndrome, and ischemia-reperfusion injury.

### Vascular Leak

Blood vessels are lined with tightly linked cells, called endothelial cells, which normally form a largely impermeable barrier. Vascular leak occurs when small blood vessels, generally a capillary or venule, become excessively leaky and release fluid and plasma protein. Vascular leak can occur under a variety of conditions, including sepsis, and can affect almost all the organ beds. The most serious effects of vascular leak include a drop in blood pressure, severe organ damage, and a lack of oxygenation of the blood when the leak is in the lung. Vascular leak disorders include, for example, any of the following disorders that have associated vascular leak: sepsis (e.g., mild or severe), septic shock, anthrax, pneumonia, acute lung injury (ALI; e.g., ventilator-induced lung injury or transfusion-related acute lung injury), acute respiratory distress syndrome (ARDS; e.g., transplant ARDS and ARDS due to burns, pancreatitis and trauma), capillary leak syndrome, ischemic brain injury, anaphylaxis, severe episodic vasculitis, ischemia-reperfusion injury, vascular leak associated with drug therapy (e.g., IL-2, IL-11, P277, ibuprofen, aspirin, salicylic acid derivatives, anagrelide, cilostazol, clopidogrel, dipyridamole, ticlopidine, anisindione, enoxaparin, heparin, pentosan polysulfate, aceclofenac, warfarin, acenocoumarol, non-steroidal anti-inflammatory drugs, selective serotonin reuptake inhibitors, and others), anthrax lethal toxin, idiopathic capillary leak syndromes, pre-eclampsia, eclampsia, hypotensive states due to sepsis, heart failure, severe burn injury, circulatory shock, trauma, infection, pulmonary aspiration of stomach contents, pulmonary aspiration of water, near drowning, burns, inhalation of noxious fumes, fat embolism, blood transfusion (TRALI, transfusion-related acute lung injury), amniotic fluid embolism, air embolism, edema, organ failure, poisoning, radiation, and inflammatory states (e.g., acute and chronic vascular rejection, pancreatitis, trauma, and vasculitis). Less common etiologies of vascular leak include genetic disorders that intermittently produce vascular leak (e.g. C1 esterase inhibitor deficiency or familial fever syndromes such as TRAPS-TNF receptor associated periodic fever syndrome), massive blood transfusion, anaphylaxis or similar hypersensitivity reactions, post-lung or post-heart-lung transplant, and ovarian hyperstimulation syndrome (e.g., as described in Garcia-Velasco et al., Curr. Opin. Obstet. Gynecol. 15: 251-256, 2003).

### Pharmaceutical Compositions

For use as treatment of human and animal subjects, the compounds of the invention can be formulated as pharmaceutical or veterinary compositions. Depending on the subject to be treated, the mode of administration, and the type of treatment desired—e.g., prevention, prophylaxis, or therapy—the compounds are formulated in ways consonant with these parameters. A summary of such techniques is found in Remington: The Science and Practice of Pharmacy, 21^{st} Edition, Lippincott Williams & Wilkins, (2005); and Encyclopedia of Pharmaceutical Technology, eds. J. Swarbrick and J. C. Boylan, 1988-1999, Marcel Dekker, New York, each of which is incorporated herein by reference.

The compounds described herein may be present in amounts totaling 1-95% by weight of the total weight of the composition. The composition may be provided in a dosage form that is suitable for intraarticular, oral, parenteral (e.g., intravenous, intramuscular), rectal, cutaneous, subcutaneous, topical, transdermal, sublingual, nasal, vaginal, intramuscular, intravesicular, intraurethral, intrathecal, epidural, aural, or ocular administration, or by injection, inhalation, or direct contact with the nasal, genitourinary, gastrointesitnal, reproductive or oral mucosa, or as part of a resuscitation fluid. Thus, the pharmaceutical composition may be in the form of, e.g., tablets, capsules, pills, powders, granulates, suspensions, emulsions, solutions, liquids, gels including hydrogels, pastes, ointments, creams, plasters, drenches, osmotic delivery devices, suppositories, enemas, injectables, implants, sprays, preparations suitable for iontophoretic delivery, or aerosols. The compositions may be formulated according to conventional pharmaceutical practice.

The compounds of the invention may be prepared and used as pharmaceutical compositions comprising an effective amount of a compound described herein and a pharmaceutically acceptable carrier or excipient, as is well known in the art. In some embodiments, the composition includes at least two different pharmaceutically acceptable excipients or carriers. Therapeutic formulations may be prepared using standard methods known in the art by mixing the active ingredient having the desired degree of purity with optional physiologically acceptable carriers, excipients or stabilizers in the form of lyophilized formulations or aqueous solutions. Acceptable carriers, include saline, or buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagines, arginine or lysine; monosaccharides, disaccharides, and other carbohydrates including glucose, mannose, or dextrins; chelating agents such as EDTA; sugar alcohols such as mannitol or sorbitol; salt-form ing counterions such as sodium; and/or nonionic surfactants.

Formulations may be prepared in a manner suitable for systemic administration or topical or local administration. Systemic formulations include those designed for injection (e.g., intramuscular, intravenous or subcutaneous injection) or may be prepared for transdermal, transmucosal, sublingual, or oral administration. The formulation will generally include a diluent as well as, in some cases, adjuvants, buffers, preservatives and the like. The compounds can be administered also in liposomal compositions or as microemulsions.

For injection, formulations can be prepared in conventional forms as liquid solutions or suspensions or as solid forms suitable for solution or suspension in liquid prior to injection or as emulsions. Suitable excipients include, for example, water, saline, dextrose, glycerol and the like. Such compositions may also contain amounts of nontoxic auxiliary substances such as wetting or emulsifying agents, pH buffering agents and the like, such as, for example, sodium acetate, sorbitan monolaurate, and so forth.

Systemic administration may also include relatively noninvasive methods such as the use of suppositories, transdermal patches, transmucosal delivery and intranasal administration. Oral administration is also suitable for compounds of the invention. Suitable forms include syrups, capsules, and tablets, as is understood in the art.

The individually or separately formulated agents of pharmaceutical compositions of the invention can be packaged together as a kit. Non-limiting examples include, but are not limited to, kits that contain, e.g., two tablets, a tablet and a powder, a suppository and a liquid in a vial, two topical creams, etc. The kit can include optional components that aid in the administration of the unit dose to patients, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

Formulations for oral use include tablets containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Two or more compounds may be mixed together in a tablet, capsule, or other vehicle, or may be partitioned. In one example, the first compound is contained on the inside of the tablet, and the second compound is on the outside, such that a substantial portion of the second compound is released prior to the release of the first compound.

Formulations for oral use may also be provided as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The liquid forms in which the compounds and compositions of the present invention can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compounds of the invention may also be formulated for sublingual administration. Sublingual administration is advantageous for active ingredients which, when given orally, are subject to a substantial first pass metabolism and enzymatic degradation through the liver, resulting in rapid metabolization and a loss of therapeutic activity related to the activity of the liver enzymes that convert the molecule into inactive metabolites, or the activity of which is decreased because of this bioconversion. The sublingual route is also capable of producing a rapid onset of action due to the considerable permeability and vascularization of the buccal mucosa. Pharmaceutical compositions of the invention may, for example, comprise a sublingual tablet with a directly compressible diluent in the form of neutral cores coated with an effective amount of bosutinib to treat or prevent vascular leak, or a tablet, capsule, pill, powder, or liquid to be administered sublingually that includes an effective amount of bosutinib to treat or prevent vascular leak.

In addition, compounds of the invention may be formulated for aerosol administration, particularly to the respiratory tract by inhalation and including intranasal administration. The compound will generally have a small particle size for example on the order of five (5) microns or less. Such a particle size may be obtained by means known in the art, for example by micronization. The active ingredient is provided in a pressurized pack with a suitable propellant, e.g., a chlorofluorocarbon (CFC), for example, dichlorodifluoromethane, trichlorofluoromethane, or dichlorotetrafluoroethane, or carbon dioxide, or other suitable gas. The aerosol may conveniently also contain a surfactant, e.g., lecithin. The dose of drug may be controlled by a metered valve. Alternatively, the active ingredients may be provided in a form of a dry powder, e.g., a powder mix of the compound in a suitable powder base, e.g., lactose, starch, and starch derivatives, e.g., hydroxypropylmethyl cellulose, and polyvinylpyrrolidine (PVP). The powder carrier will form a gel in the nasal cavity. The powder composition may be presented in unit dose form for example in capsules or cartridges of e.g., gelatin or blister packs from which the powder may be administered by means of an inhaler.

Aerosol formulations typically include a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device, e.g., a single dose nasal inhaler or an aerosol dispenser fitted with a metering valve which is intended for disposal after use. Where the dosage form comprises an aerosol dispenser, it will contain a propellant, which can be a compressed gas, e.g., compressed air or an organic propellant, e.g., fluorochlorohydrocarbon. The aerosol dosage forms can also take the form of a pump-atomizer.

### Automatic Injection Devices

An automatic injection device of the invention is a device that enables intramuscular or subcutaneous administration of a dosage of medicament. Generally, the medicament is stored as a liquid formulation which is then injected intramuscularly. An advantage of automatic injectors is that they contain a measured dosage of a liquid medicament in a sealed sterile cartridge. As such, automatic injectors allow for quick and simple IM injection of a liquid medicament in emergency situations without the need for measuring dosages. Another advantage of automatic injectors is that the administration of the medicament is accomplished without the user initially seeing the hypodermic needle through which the medicament is delivered, and without requiring the user to manually force the needle into the patient. This is particularly advantageous when the medicament is being self-administered.

Automatic injection devices known in the art include, for example, those disclosed in U.S. Pat. No. 2,832,339; U.S. Pat. No. 5,295,965; U.S. Pat. No. 6,641,561; U.S. Pat. No. 7,449,012; and U.S. Pat. No. 7,794,432; all of which are incorporated herein in their entirety. In a preferred embodiment, the automatic injection device of the invention includes an effective amount of bosutinib for treating or preventing acute vascular leak.

### Dosage

The dosage and the timing of administering pharmaceutical compositions and/or compounds of the invention depends on various clinical factors including the overall health of the subject and the severity of the symptoms of the vascular leak. The invention includes, for example, the use of bosutinib to treat, prevent or reduce conditions associated with acute vascular leak, or the risk of developing acute vascular leak, in a subject. Bosutinib can be administered at anytime, for example, after diagnosis or detection of a vascular leak or a condition associated with vascular leak (e.g., using the diagnostic methods known in the art or described herein), or for prevention of a vascular leak disorder in subjects that have not yet been diagnosed with a vascular leak disorder but are at risk of developing such a disorder (e.g., subjects suffering from or being treated for sepsis), after a risk of developing a vascular leak disorder is determined.

Compounds of the present invention can be formulated and administered in a variety of ways, e.g., those routes known for specific indications, including, but not limited to, topically, orally, subcutaneously, bronchial injection, intravenously, intracerebrally, intranasally, transdermally, intraperitoneally, intramuscularly, intrapulmonary, vaginally, rectally, intraarterially, intralesionally, parenterally, intraventricularly in the brain, or intraocularly. For example, the compound can be in the form of a tablet, capsule, or liquid for oral or sublingual administration; or a liquid for intramuscular, intravenous, subcutaneous or administration; a polymer or other sustained release vehicle for local administration; an ointment, cream, gel, liquid, or patch for topical administration. Continuous systemic infusion or periodic injection of the compound can be used to treat or prevent the disorder. Treatment can be continued for a period of time ranging from 1 day through the lifetime of the subject, more preferably 1 to 100 days, and most preferably 1 to 20 days and most preferably, until the symptoms of vascular leak are reduced or removed. Dosages vary depending on the compound and the severity of the condition. Compounds of the invention can be administered continuously by infusion, using a constant- or programmable-flow implantable pump, or by periodic injections. Sustained release systems can also be used. Semipermeable, implantable membrane devices are also useful as a means for delivering compounds of the invention in certain circumstances. In another embodiment, the compound is administered locally, e.g., by inhalation, and can be repeated periodically.

Optionally, but preferably, the formulation contains a pharmaceutically acceptable salt, preferably sodium chloride, and preferably at about physiological concentrations. Optionally, the formulations of the invention can contain a pharmaceutically acceptable preservative. In some embodiments the preservative concentration ranges from 0.1 to 2.0%, typically v/v. Suitable preservatives include those known in the pharmaceutical arts. Benzyl alcohol, phenol, m-cresol, methylparaben, and propylparaben are preferred preservatives. Optionally, the formulations of the invention can include a pharmaceutically acceptable surfactant. Preferred surfactants are non-ionic detergents. Preferred surfactants include Tween 20 and pluronic acid (F68). Suitable surfactant concentrations are 0.005 to 0.02%.

The dosage of compounds of the invention will depend on other clinical factors such as weight and condition of the subject and the route of administration of the compound. For treating subjects, between approximately 0.1 mg/kg to 500 mg/kg body weight of the compound can be administered. A more preferable range is 1 mg/kg to 150 mg/kg body weight with the most preferable range being from 1 mg/kg to 50 mg/kg body weight. Depending upon the half-life of the compound in the particular subject, the compound can be administered between several times per day to once a week. The methods of the present invention provide for single as well as multiple administrations, given either simultaneously or over an extended period of time.

The dosage required depends on the choice of the route of administration; the nature of the formulation; the nature of the subject's illness; the subject's size, weight, surface area, age, and sex; other drugs being administered; and the judgment of the attending physician. Wide variations in the needed dosage are to be expected in view of the variety of polypeptides and fragments available and the differing efficiencies of various routes of administration. For example, oral administration would be expected to require higher dosages than administration by intravenous injection. Variations in these dosage levels can be adjusted using standard empirical routines for optimization as is well understood in the art. Administrations can be single or multiple (e.g., 2-, 3-, 6-, 8-, 10-, 20-, 50-, 100-, 150-, or more). Encapsulation of the compound in a suitable delivery vehicle (e.g., polymeric microparticles or implantable devices) may increase the efficiency of delivery, particularly for oral delivery.

### Combination Therapies

In general, for use in treatment, the compounds described herein may be used alone, as mixtures of two or more compounds, or in combination with other pharmaceuticals and therapies. The compounds and pharmaceutical compositions of the invention can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder, or they may achieve different effects (e.g., control of any adverse effects). It is contemplated that the therapies, when applied in combination, may have additive effects, i.e., the effect achieved when the active ingredients are used together is greater than either active ingredient provides when used alone.

The combination therapy may provide “synergy” and prove “synergistic,” i.e., the effect achieved when the active ingredients used together is greater than the sum of the effects that results from using the compounds separately. A synergistic effect may be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect may be attained when the compounds, agents, and/or treatments are administered or delivered sequentially, e.g., by different injections in separate syringes. In general, during alternation therapy, an effective dosage of each active ingredient is administered sequentially, i.e., serially, whereas in combination therapy, effective dosages of two or more active ingredients are administered together. Suitable dosages for any of the above co-administered agents are those presently used and may be lowered due to the combined action (synergy) of a compound of the present invention and other co-administered agents or treatments.

An example of other pharmaceuticals to combine with the compounds described herein would include pharmaceuticals for the treatment of the same indication. Another example of a potential pharmaceutical to combine with the compounds described herein would include pharmaceuticals for the treatment of different yet associated or related symptoms or indications. Depending on the mode of administration, the compounds will be formulated into suitable compositions to permit facile delivery. Each compound of a combination therapy may be formulated in a variety of ways that are known in the art. For example, the first and second agents of the combination therapy may be formulated together or separately. Desirably, the first and second agents are formulated together for the simultaneous or near simultaneous administration of the agents.

Thus, in various embodiments, the methods, pharmaceutical compositions, devices, and/or kits of the present invention can be provided in conjunction (e.g., before, during, or after) with additional vascular leak therapies to prevent or reduce a vascular leak disorder. Methods, pharmaceutical compositions, devices, and/or kits of the invention can be used in combination with one or more compounds known in the art for the treatment of vascular leak. Examples of such compounds include a Src kinase inhibitor (e.g., imatinib, PP1, PP2, sunitinib, or SKS-927), a Rho kinase inhibitor (e.g., Y-27632 or fasudil), a soluble epoxide hydrolase inhibitor (e.g., 12-(3-adamantan-1-yl-ureido)-dodecanoic acid butyl ester or 1-adamantan-3-(5-(2-(2-ethyl-ethoxy)ethoxy)pentyl)urea), a TREM1 inhibitor (e.g., recombinant TLT-1, LR17 peptide, Inotrem MOTREM peptide, or SignaBlock SCHOOL peptide), a TNF-α inhibitor (e.g., infliximab, adalimumab, etanercept, or ADZ9773), an antibiotic, drotrecogin alfa, a corticosteroid, and vasopressin. Treatment therapies that can be used in combination with the methods, compounds, pharmaceutical compositions, devices, and/or kits of the invention include but are not limited to and/or the administration of a mechanical ventilation device, surgical drainage of infected fluid collections, fluid replacement, and appropriate support for organ dysfunction, including, for example, hemodialysis in kidney failure, mechanical ventilation in pulmonary dysfunction, transfusion of blood plasma, platelets, and coagulation factors to stabilize blood coagulation, and drug and fluid therapy for circulatory failure. Additional such therapies can include activated protein C therapy (drotrecogin alfa), corticosteroid treatment, vasopressin, inhibitors of MLC kinase, inhibitors of VEGF (e.g., avastin), inhibitors of PIGF, inhibitors of NFκB (e.g., panepoxydone), inhibitors of TNF-α, inhibitors of IL-1, IL-6, Ang-2 antagonists, and inhibitors of TGF-β. Desirably, bosutinib can be formulated alone or in combination with any additional vascular leak therapies, either described herein or known in the art.

### Companion Diagnostics

The methods of the invention may be used in combination with one or more companion diagnostic tests for vascular leak conditions. Exemplary companion diagnostics useful as measures reflective of vascular leak (e.g., exudative vascular leak and/or acute vascular leak) include coordinated monitoring of blood pressure, edema (e.g., by conducting physical examinations to, for example, measure ankle thickness and/or tissue swelling) and severity of organ dysfunction (e.g., by conducting Sequential Organ Failure Assessments (SOFA) via standardized laboratory tests and clinical data). Examples of SOFA scores useful in the methods of the present invention include measuring: bilirubin, creatinine, platelet count, partial pressure of oxygen, fraction of inhaled O₂, the Glasgow Coma Scale, and/or hypotension. Additional methods that may be useful for diagnosing vascular leak conditions include examining a subject's health history, immunohistochemical staining of tissues, or performing one or more laboratory tests, such as those described herein. The results of such companion diagnostic tests can be used to identify candidates likely to be in need of anti-vascular leak therapy and to monitor the efficacy of the compound or composition of the present invention administered to the subject and therefore raise or lower the dosage as appropriate. The diagnostic methods described herein can be used individually or in combination with any other diagnostic method described herein for a more accurate diagnosis of the presence or severity of vascular leak (e.g., acute vascular leak).

### Kits

The present invention relates to a kit for conveniently and effectively carrying out the methods in accordance with the present invention. In general, the pharmaceutical pack or kit comprises one or more containers filled with one or more of the ingredients of the pharmaceutical compositions of the invention. Such a kit preferably includes a number of unit dosages, and may also include a card having the dosages oriented in the order of their intended use. A memory aid can be provided, for example, in the form of numbers, letters, or other markings or with a calendar insert, designating the days in the treatment schedule in which the dosages can be administered. Optionally associated with such container(s) can be a notice in the form prescribed by a governmental agency regulating the manufacture, use or sale of pharmaceutical products, which notice reflects approval by the agency of manufacture, use or sale for human administration.

The following examples are intended to illustrate, rather than limit, the invention.

### EXAMPLES

### Example 1

### In Vitro Vascular Stabilization and Protection by Bosutinib

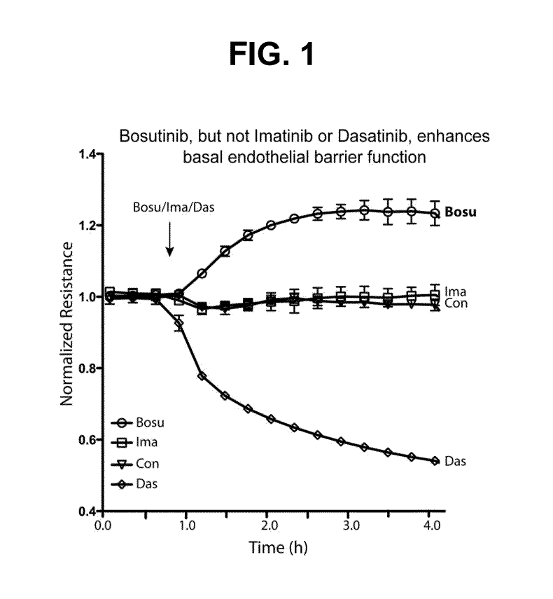

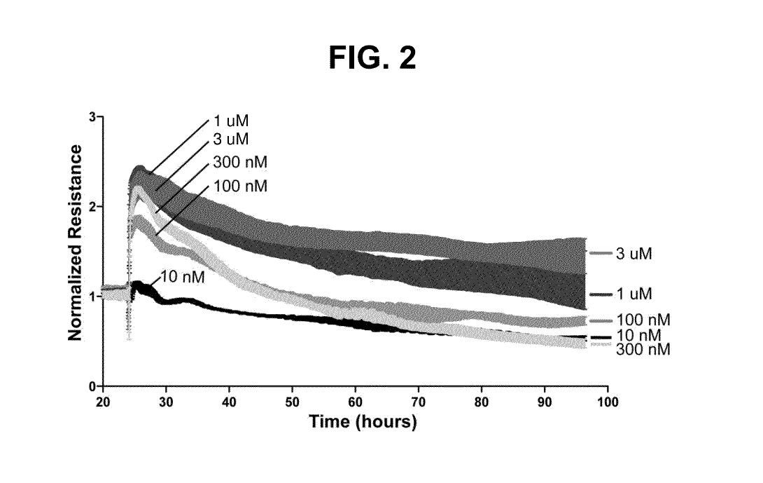

Our research focuses on the vascular endothelium, the monolayer of endothelial cells that function collectively to generate the critical barrier that separates the blood circulation and the underlying tissues. Angiopoietins are growth factors that can influence both endothelial cell growth and barrier properties. In in vitro studies designed to dissect signaling pathways downstream of Angiopoietin-2 (Ang-2) in endothelial cells we employed imatinib, dasatinib and bosutinib as pharmacologic tools to interrupt Src/Abl signaling. In the course of this work, we made the entirely unexpected finding that in control experiments bosutinib, but not imatinib or dasatinib (FIG. 1) profoundly enhanced base-line barrier properties in otherwise untreated endothelial monolayers. Imatinib had no effect and dasatinib reduced endothelial barrier function. Bosutinib showed an unprecedented capacity to elicit sustained barrier enhancement effects in a dose-dependent manner, lasting as long as 72 hours (the longest experiments conducted thus far; FIG. 2).

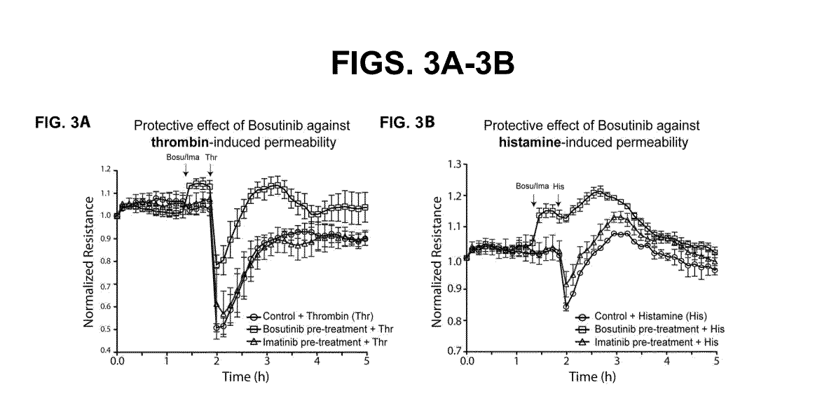

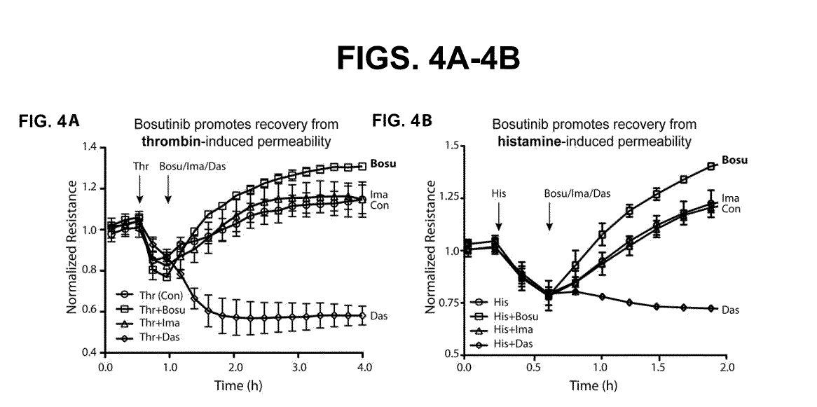

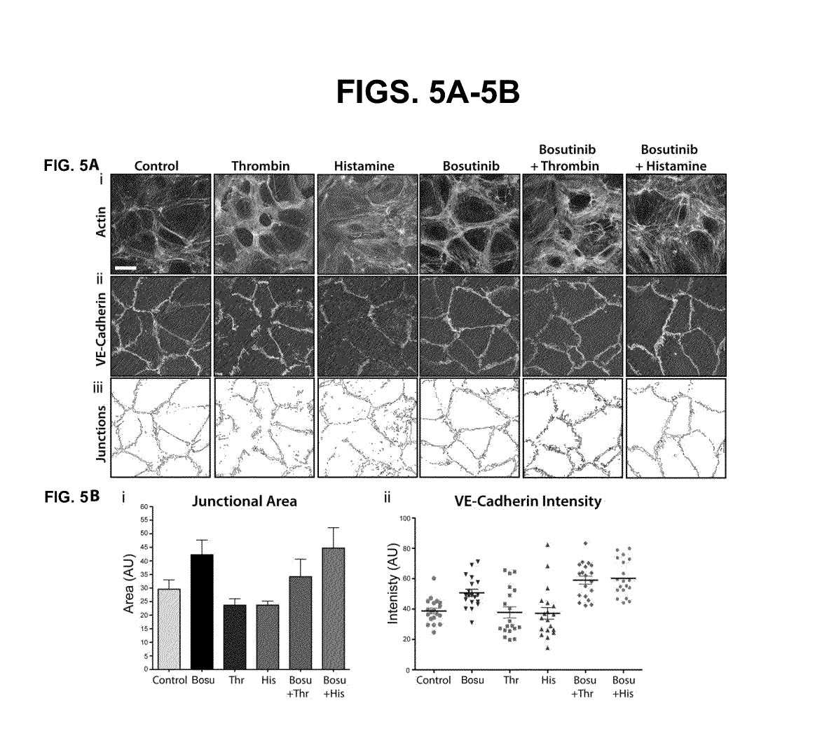

Since endothelial barrier function derives from the efficacy of a monolayer of individual endothelial cells to act as a collective/cohesive structure, our finding suggested that bosutinib can act directly on vascular endothelial cells to stabilize their collective integrity. Based on these findings, we tested the effects of bosutinib and imatinib pretreatment on endothelial responses to diverse physiological/pathological barrier disrupting (i.e., leak promoting) agents. Our studies showed that bosutinib pretreatment, but not imatinib pretreatment, conferred profound protection against a range of pro-leak stimuli (e.g., thrombin, histamine, Ang-II and TNF-α) (FIGS. 3A and 3B). Furthermore we found that “post-treatment” with bosutinib following initial stimulation with pro-leak agonist caused accelerated recovery of the endothelial barrier (FIGS. 4A and 4B). Imatinib had no effect and dasatinib promoted exacerbated and sustained barrier dysfunction. Our imaging-based analysis showed that the effects were associated with a decrease in actin stress fibers in endothelial cells, and an increase in VE-cadherin and cortical actin at the intercellular junctions, which is consistent with enhanced intercellular integrity (FIGS. 5A and 5B).

### Example 2

### Benchmarking Bosutinib Against Other Barrier Protection Agents

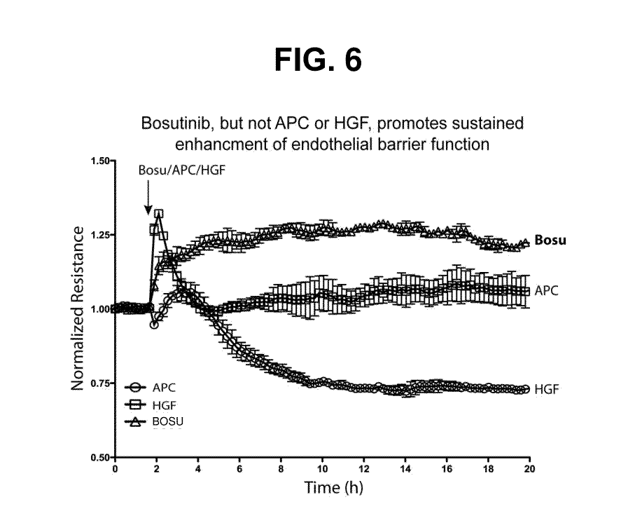

A range of physiologic and pharmacologic agents have been well characterized for their barrier stabilizing properties including hepatocyte growth factor, activated protein C (APC), sphingosine-1 phosphate, adrenomedulin, Y-27632, and fasudil. Several of these have shown efficacy at preventing leak in preclinical animal models and APC was temporarily FDA approved for treatment of septic shock, based in part on its ability to stabilize the vascular barrier. Benchmarked against APC and many of the other “classic” barrier protection agents, we find that bosutinib exhibits unprecedented efficacy and duration of action (FIG. 6). Based on these and other mechanistic findings (described below), we propose to develop bosutinib as novel vascular barrier protective either mono- or combination-therapy for wide ranging vascular leak pathologies (VLP). These include, but are not limited, to both relatively acute VLP such as septic shock, acute lung injury (ALI), acute respiratory distress syndrome (ARDS), ventilator-induced lung injury (VILI), transfusion-related acute lung injury (TRALI), ischemia-reperfusion injury, stroke, severe burn injury, circulatory shock, anthrax lethal toxin induced vascular leak, capillary leak syndrome, and anaphalaxis.

### Example 3

### Bosutinib Alters Endothelial Barrier Function in a Src-Independent Manner

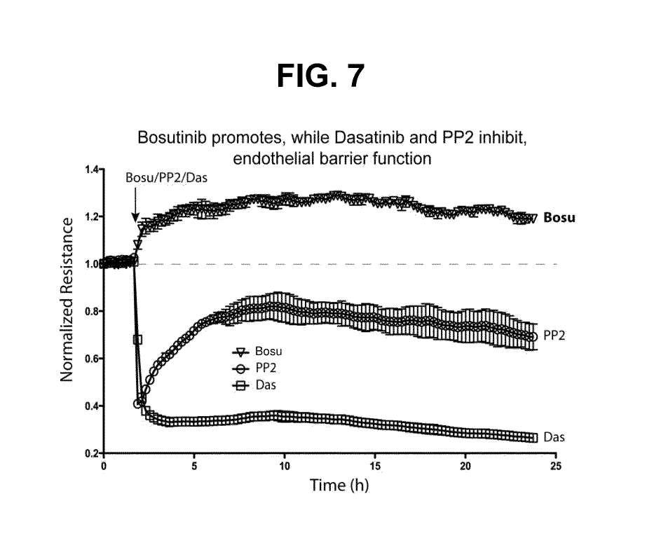

Bosutinib, imatinib, and dasatinib all share similar efficacy against Src and Abl kinases in the low nanomolar range. However, several global target analyses have revealed a multitude of both overlapping and disparate kinases inhibited by each of these molecules in the low-mid nanomolar range (Bantscheff et al., Nat. Biotechnol., 25: 1035-1044, 2007; Rix et al., Leukemia, 23: 477-485, 2009; Boschelli et al., Eur. J. Cancer, 46: 1781-1789, 2010). Thus, our unintended and unexpected demonstration of differential effects of these molecules on endothelial cell barrier function establishes that bosutinib alters endothelial barrier function in a manner that is either independent, or at least not primarily dependent, on the intuitive targets (i.e., Src and Abl). A corollary to this is that the effects of bosutinib depend on a unique (as yet undefined) target or constellation of targets (which may or may not include Src and Abl). This conclusion is further supported by our finding that alternate Src-family inhibitors, including PP2 (with specificity for p56Lck, p59Fyn, Hck and Src) and dasatinib, not only failed to increase, but in fact greatly reduced, endothelial barrier function (FIG. 7). Our assessment of the identified bosutinib versus imatinib and dasatinib targets, with emphasis on those known to be involved in vascular barrier, junction, and cytoskeletal remodeling, suggests a range of potential functional targets that might be involved in the barrier stabilizing effect of bosutinib including: calcium-calmodulin kinase (CAMK) 2δ, CAMK2γ, EphrinA2, EphrinB2, EphrinB4, LIMK1, ERK1/2, p38 MAPK, CSK, STE20 and ROCK2.

### Example 4

### Bosutinib has a Dual Protective Mechanism In Vivo

Existing evidence demonstrates that, during any severe tissue trauma (e.g., burn injury), systemic inflammatory responses, including cytokine storm, can develop that are quite similar to those seen in sepsis (Quaid et al., Arch. Surg. 134: 1376-1371, 1999; Nuytinck et al., Arch. Surg. 123: 1519-1524, 1988; Dahiya, Front. Biosci. 12: 4962-4967, 2009). These both directly cause some vascular leak and cause inappropriate activation of white blood cells, mostly neutrophils, that in turn can damage endothelium and cause a great deal more vascular leak. Our preliminary data indicates that such neutrophil-mediated vascular damage is a central component of the mechanism driving acute vascular leak in most pathologic settings. We have found that bosutinib may have a dual protective mechanism in vivo: (1) direct endothelial barrier protection (as described in Examples 1-3), and (2) inhibition of neutrophil activation by blocking the Src-family kinases Fgr and Hck, which have been shown to regulate important tissue damaging functions of these cells (Moscai et al., J. Immunol. 162: 1120-1126, 1999).

### Example 5

### Synergistic Effects of Combining Bosutinib with Classic Barrier Stabilizing Agents

Many physiologic hormone and pharmacologic small molecule agents have been identified with vascular barrier enhancing/protecting capacity, which are being explored as potential vascular leak therapies. Such “classic barrier protectors” include, but are not limited to, adrenomedulin, hepatocyte growth factor, angiopoietin-1, sphingosine-1-phosphates, beta-agonists (e.g., isoproterenol), activated protein C (APC), O-methyl-cAMP, fasudil, and Y27632. It is likely that the mechanisms of barrier enhancement/protection for these are at least partially distinct from that of bosutinib. Thus, combined use of bosutinib with at least a subset of classic barrier protectors may have additive or synergistic barrier protection capacity. We will conduct systematically, pairwise tests of bosutinib with wide ranging classic barrier protector agents to assess this possibility in vitro ECIS studies, for example.

### Example 6

### Combined Barrier Protection and Colloid Resuscitation

Currently, the primary line of therapy for sepsis and other acute VLP is fluid resuscitation (i.e., replenishment) to restore the necessary circulating blood volume and pressure. During vascular leak, fluid loss is accompanied by loss of plasma proteins that are critical to maintain the intravascular osmotic gradient/ontonic pressure. Thus, fluid resuscitation with addition of colloid supplements (e.g., albumin or gelatin) should theoretically have improved benefit by restoring the intravascular colloidal osmotic gradient/ontonic pressure. Although, in practice, some benefits of colloidal resuscitation are seen in human patients and rodents, these fall well short of the theoretical expectations. This may be due to the idea that, in the absence of a correction in vascular leak, the added colloids eventually move into the tissue, where they might even exacerbate tissue edema and circulatory dysfunction. Thus, we propose to develop a novel approach whereby “barrier leak correction” (via, e.g., bosutinib, classic barrier protectors, or a combination thereof) and colloidal resuscitation are provided in combination (e.g., simultaneously or sequentially). We predict that such a unique regime may have profoundly cooperative benefits, especially in the most extreme settings of shock. To develop this approach, we will implement an established, advanced pre-clinical Intensive Care Unit Model (ICU), whereby animals are treated with fluid resuscitation and antibiotics to mimic clinical ICU settings, while parameters of cardiac output, tissue perfusion and oxygenation and vascular permeability can be monitored in real-time. We will examine the efficacy of “barrier leak correction” in this setting alone or in combination with colloid (e.g., 4% albumin) resuscitation regimes, whereby “barrier leak correction” is administered at varied intervals before, concomitant with, or at variable intervals after resuscitation. We expect that the positive effects of colloid resuscitation will be additive or synergistic with those of barrier leak correction. Based on our extensive in vitro results we would expect that administration of barrier leak correction shortly before or concomitant with resuscitation (conditions that can easily be achieved clinically) will be most effective, whereas administration of barrier leak correction at progressively later intervals following resuscitation will be progressively less effective.

### Example 7

### Bosutinib for Sepsis in Rodent Models

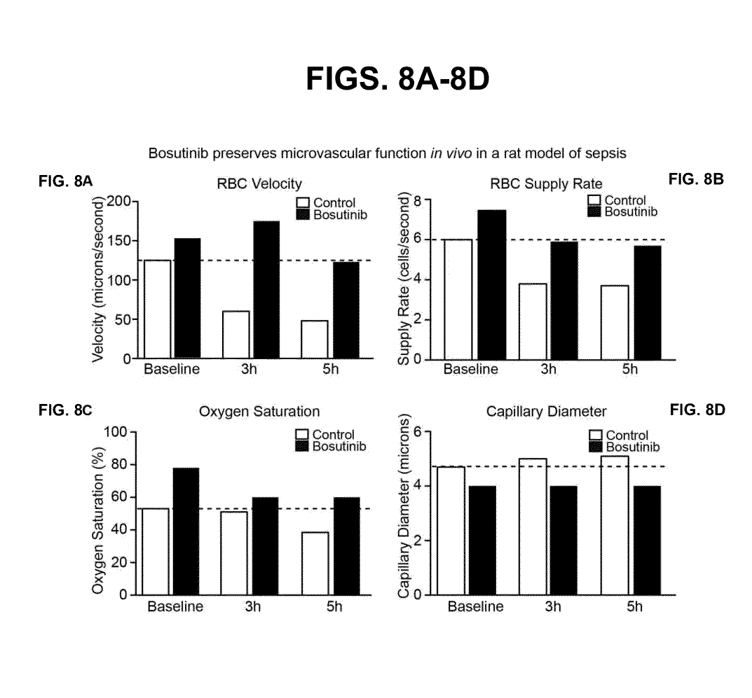

Our in vitro studies show that bosutinib can protect and rescue vascular barrier breakdown associated with sepsis pathology in vivo. To demonstrate initial in vivo proof-of-principle of this effect, we will conduct studies in three distinct established models of sepsis. In the first model, the bacterial-derived toxin lipopolysaccharide (LPS) will be administered systemically in mice to produce a well-controlled sepsis-like response. In the second model, a true polymicrobial sepsis will be generated in mice via the cecal ligation puncture (CLP) protocol. In the third model, polymicrobial sepsis will be generated in rats in an advanced model that mimics treatment regimes that humans receive in clinical Intensive Care Units, whereby animals are treated with fluid resuscitation and antibiotics while parameters of cardiac output, tissue perfusion and oxygenation and vascular permeability can be monitored in real-time. These models are complementary and progress from being highly controlled but less realistic to being highly realistic but more variable. In all of these models, we will investigate directly vascular integrity (through electron and fluorescence confocal microscopy) and barrier function (through Evan's Blue and colloidal carbon tracer studies). These will be done in concurrence with survival studies and, where possible (e.g., the rat model), with real-time measurement of tissue perfusion and oxygenation and vascular permeability. We will examine the effects of systemic administration bosutinib at various time points before and after initiation of sepsis. We expect that bosutinib will be effective in all models when administered shortly before or after sepsis onset and that its efficacy will diminish to some degree when administered at greater time points are which disease severity is heightened. Initial pilot studies in the rat polymicrobial sepsis model demonstrated that leak-affected parameters including microvascular flow (FIGS. 8A and 8B) and oxygen saturation (FIG. 8C) were greatly decreased in control (vehicle-treated) septic rats, but effectively preserved in bosutinib-treated septic rats. Capillary diameter was unaltered in either control or bosutinib-treated septic rats (FIG. 8D), indicating that the benefits conferred by bosutinib treatment were unrelated to vasodilation and were instead attributable to correction of vascular leak.

### Example 8

### Bosutinib for Additional Vascular Leak Indications

We propose that bosutinib may serve as a novel therapy for wide-ranging vascular leak pathologies (VLP) in addition to sepsis. These include, but are not limited, to relatively acute VLP such as acute stroke, ischemia-reperfusion injury, anaphylaxis, lung injury (ALI), acute respiratory distress syndrome (ARDS), ventilator-induced lung injury (VILI), transfusion-related acute lung injury (TRALI), circulatory shock, anthrax lethal toxin induced vascular leak, capillary leak syndrome, and burn injury. Thus, as additional examples, we will investigate the effects of bosutinib in surgical models of hind limb and cerebral (i.e., stroke) ischemia-reperfusion injury, whereby murine blood vessels will surgically ligated for a fixed amount of time and then reopened by removing the ligation. We will also test an immunological model of anaphylaxis, whereby mice will be sequentially sensitized and then challenged with immunogen and adjuvant. We will determine the effects of administration of bosutinub at variable intervals prior to application of the prescribed manipulations in each model. We predict that bosutinib will be effective at reducing leak in all models and most effective when administered before or shortly after application of these manipulations. Importantly, the possibility of pretreatment with bosutinib has clinical relevance for ischemic-reperfusion injury. In this case, patients often arrive at the emergency room with a blocked vessel (e.g., by a blood clot; which is modeled by surgical ligation). Actions are then taken clinically to remove the blockage (which is modeled by removing the ligation; “reperfusion”) to preserve the effected tissues. Reperfusion paradoxically also causes extensive exacerbated damage to the vessels and tissues before healing begins. Such reperfusion injury could potentially be prevented in part by administration of bosutinib as a priming therapy prior to the necessary clot-removing therapy.

### Example 9

### Clinical Trials for Bosutinib in Treating Human Septic Shock

Bosutinib has a low toxicity profile and is currently FDA approved for treatment of imatanib-resistant CML patients. Bosutinib is therefore poised for fast track trials in areas of critical unmet need such as sepsis. Thus, following success in pre-clinical rodent models (see Example 6), we will conduct pilot studies in humans. We will recruit adult patients (>18 years old) presenting to the Emergency Department or ICU who meet previously established criteria for septic shock (including a systolic blood pressure <90 mmHg after a 20 cc/kg fluid challenge). Standard of care includes administration of antibiotics, fluid resuscitation, and monitoring of blood pressure and organ function. We will randomize a cohort of patients to receive either standard care or standard care with concomitant administration of either a single dose of bosutinib during fluid resuscitation or multiple doses of bosutinib administered both during initial resuscitation and at varied intervals thereafter. We predict that patients receiving bosutinib will recover blood pressure and organ function more quickly, have better survival rates and retain better long-term quality of life. Additionally, we will perform similar studies whereby patients receive colloidal (e.g., 4% albumin) resuscitation and bosutinib. We expect that these conditions will generate further improvements in survival and long-term quality of life.

### OTHER EMBODIMENTS

All publications, patents, and patent applications mentioned in the above specification are hereby incorporated by reference to the same extent as if each individual publication, patent or patent application was specifically and individually indicated to be incorporated by reference in its entirety. Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A method of treating or preventing acute vascular leak in a subject in need thereof, wherein said method comprises contacting an effective amount of bosutinib with said subject, and wherein said acute vascular leak is associated with a condition selected from the group consisting of: ischemic brain injury, septic shock, anthrax lethal toxin, anaphylaxis, trauma, severe episodic vasculitis, acute lung injury, acute respiratory distress syndrome, severe burn injury, circulatory shock, capillary leak syndrome, and ischemia-reperfusion injury.

2. (canceled)

3. The method of claim 1, wherein
said ischemic brain injury is stroke,
said acute lung injury is ventilator-induced lung injury or transfusion-related acute lung injury, or
said trauma is selected from the group consisting of: severe tissue trauma, head trauma, lung trauma, cardiac trauma, and vascular trauma.

4-5. (canceled)

6. The method of claim 1, wherein said subject is at risk for acute vascular leak associated with a condition selected from the group consisting of: ischemic brain injury, septic shock, anthrax lethal toxin, anaphylaxis, trauma, severe episodic vasculitis, acute lung injury, acute respiratory distress syndrome, severe burn injury, circulatory shock, capillary leak syndrome, and ischemia-reperfusion injury.

7. The method of claim 1, wherein said bosutinib is administered by autoinjection, as a component of a resuscitation fluid, sublingually, or by inhalation.

8-10. (canceled)

11. The method of claim 1, further comprising administering a second therapeutic agent.

12. The method of claim 11, wherein said second therapeutic agent is selected from the group consisting of: a Src inhibitor, a Rho kinase inhibitor, a soluble epoxide hydrolase inhibitor, a TREM1 inhibitor, a TNF-α inhibitor, an antibiotic, a corticosteroid, vasopressin, a mechanical ventilation device, surgical drainage of infected fluid collections, fluid replacement, support for organ dysfunction, activated protein C therapy, corticosteroid treatment, vasopressin, an MLC kinase inhibitor, a VEGF inhibitor, a PIGF inhibitor, an NFκB inhibitor, an IL-1 inhibitor, an IL-6 inhibitor, an Ang-2 antagonist, and a TGF-β inhibitor.

13. The method of claim 12, wherein
said Src inhibitor is selected from the group consisting of: Nilotinib, Ponatinib, Bafetinib, imatinib, dasatinib, PP1, PP2, sunitinib, and SKS-927,
said Rho kinase inhibitor is Y-27632 or fasudil,
said soluble epoxide hydrolase inhibitor is 12-(3-adamantan-1-yl-ureido)-dodecanoic acid butyl ester (AUDA-BE), 1-adamantan-3-(5-(2-(2-ethyl-ethoxy)ethoxy)pentyl)urea (compound 950), or any combination thereof,
said TREM1 inhibitor is recombinant TREM-like transcript 1 (TLT-1), LR17 peptide, Inotrem MOTREM peptide, or SignaBlock SCHOOL peptide,
said TNF-α inhibitor is infliximab, adalimumab, etanercept, or ADZ9773,
said support for organ dysfunction is selected from the group consisting of: hemodialysis, mechanical ventilation, transfusion of blood plasma, platelets, and coagulation factors, and drug and fluid therapy,
said activated protein C therapy is drotrecogin alfa,
said VEGF inhibitor is avastin, or
said NFκB inhibitor is panepoxydone.

14-27. (canceled)

28. A method of treating vascular leak in a subject identified as likely to benefit from bosutinib treatment by monitoring one or more measures reflective of vascular leak, said method comprising
administering an effective amount of bosutinib to said subject if said one or more measures indicate increased vascular leak, thus treating said vascular leak in said subject.

29. The method of claim 28, wherein said one or more measures reflective of vascular leak comprises measuring blood pressure, edema, severity of organ dysfunction, red blood cell (RBC) velocity, RBC supply rate, percent oxygen saturation, capillary diameter, or any combination thereof.

30. The method of claim 29, wherein
said edema is measured by conducting physical examination of ankle thickness or tissue swelling, or
said severity of organ dysfunction is measured by conducting one or more Sequential Organ Failure Assessments (SOFA).

31. (canceled)

32. The method of claim 28, wherein said indication of increased vascular leak comprises a decrease in blood pressure, an increase in edema, an increase in severity of organ dysfunction, a decrease in RBC velocity, a decrease in RBC supply rate, decrease in oxygen saturation, or any combination thereof.

33. The method of claim 32, wherein said increase in edema comprises increased ankle thickness or increased tissue swelling.

34. The method of claim 28, further comprising monitoring said one or more measures reflective of vascular leak after said administration, wherein a reduction of said indication of increased vascular leak indicates treatment of said vascular leak.

35. The method of claim 28, wherein said bosutinib has not been previously administered to said subject to treat said vascular leak, or wherein said bosutinib has been previously administered to said subject to treat said vascular leak.

36. (canceled)

37. The method of claim 28, wherein said vascular leak is an acute vascular leak.

38. The method of claim 37, wherein said acute vascular leak is associated with a condition selected from the group consisting of: ischemic brain injury, stroke, septic shock, anthrax lethal toxin, anaphylaxis, trauma, severe episodic vasculitis, acute lung injury, ventilator-induced lung injury, transfusion-related acute lung injury, acute respiratory distress syndrome, severe burn injury, circulatory shock, capillary leak syndrome, and ischemia-reperfusion injury.

39. The method of claim 28, wherein said bosutinib is administered by autoinjection, as a component of a resuscitation fluid, sublingually, or by inhalation.

40-42. (canceled)

43. A pharmaceutical composition in unit dosage form comprising an effective amount of bosutinib for treating or preventing acute vascular leak, wherein said unit dosage form comprises:
an automatic injection device comprising a pre-loaded charge of medicament for automatically self-administering said medicament upon actuation thereof, wherein said medicament comprises said bosutinib;
a resuscitation fluid comprising said bosutinib;
a tablet to be administered sublingually, said tablet comprising said bosutinib;
a liquid to be administered sublingually, said liquid comprising said bosutinib;
a powder to be administered sublingually, said liquid comprising said bosutinib; or
an aerosol comprising said bosutinib.

44-48. (canceled)

49. The pharmaceutical composition of claim 43, wherein said acute vascular leak is associated with a condition selected from the group consisting of: ischemic brain injury, stroke, septic shock, anthrax lethal toxin, anaphylaxis, trauma, severe episodic vasculitis, acute lung injury, ventilator-induced lung injury, transfusion-related acute lung injury, acute respiratory distress syndrome, severe burn injury, circulatory shock, capillary leak syndrome, and ischemia-reperfusion injury.

50. A kit for treating or preventing acute vascular leak in a subject in need thereof, comprising an effective amount of bosutinib and instructions for administering said bosutinib to said subject according to the method of claim 1.

51. The kit of claim 50, further comprising an automatic injection device, a resuscitation fluid, a tablet to be administered sublingually, a liquid to be administered sublingually, a powder to be administered sublingually, or an aerosol,
wherein said automatic injection device, resuscitation fluid, tablet to be administered sublingually, liquid to be administered sublingually, powder to be administered sublingually, or aerosol comprises said bosutinib.

52-56. (canceled)

57. The kit of claim 50, wherein said acute vascular leak is associated with a condition selected from the group consisting of: ischemic brain injury, stroke, septic shock, anthrax lethal toxin, anaphylaxis, trauma, severe episodic vasculitis, acute lung injury, ventilator-induced lung injury, transfusion-related acute lung injury, acute respiratory distress syndrome, severe burn injury, circulatory shock, capillary leak syndrome, and ischemia-reperfusion injury.

58-59. (canceled)
